(19) United States
(12) Patent Application Publication
(10) Pub. No.: US 2025/0076290 A1
(43) Pub. Date: Mar. 6, 2025

(54) ASSAY METHOD USING MAGNETIC PARTICLES

(71) Applicant: HORIBA ABX SAS, MONTPELLIER (FR)

(72) Inventors: Aurélien DAYNES, MONTPELLIER (FR); Nevzat TEMUROK, MONTPELLIER (FR)

(73) Assignee: HORIBA ABX SAS, MONTPELLIER (FR)

(21) Appl. No.: 18/722,357

(22) PCT Filed: Dec. 13, 2022
(86) PCT No.: PCT/FR2022/052334

§ 371 Date: Jun. 20, 2024

(30) Foreign Application Priority Data
Dec. 21, 2021 (FR) FR2114135

(51) Int. Cl. G01N 33/543

(52) U.S. Cl. CPC G01N 33/54333; G01N 2470/04

(57) ABSTRACT

Method for assaying a target analyte in a biological sample in liquid medium, comprising: contacting the sample with first magnetic particles bearing a first receptor specific to a first analyte site of attachment to form first complexes; applying a first magnetic field to locally combine the formed complexes formed and optionally to agglomerate interfering complexes to form interfering aggregates; negating the applied magnetic field; adding second magnetic particles to a liquid medium that bear a second receptor specific to a second analyte site of attachment; measuring a first quantity of interfering aggregates; applying a second magnetic field to form second complexes; measuring a second quantity of the collective amount of interfering aggregates and second complexes to determine an amount of formed second complexes as a function of the first quantity, and deducing the amount of analyte present in the sample and, optionally, the amount of interfering analyte.

The invention relates to a method for assaying a target analyte in a liquid sample, and to an assay device intended to carry out this method.

More generally, the invention relates to the field of assaying molecules of interest soluble in a liquid medium. In particular, it relates to the field of immunoassay of proteins, for example in a whole blood sample.

The prior art proposes assay methods that use magnetic particles for capturing and/or extracting analytes present in a liquid sample. For this purpose, the magnetic particles bear one or more receptor(s) specific to the target analyte, which enables them to bond to the analyte to form complexes. Afterwards, the complexes can be captured by application of a magnetic field.

The applications of assays based on magnetic particles are broad. Some aim applications in oncology, cf., for example, the document Xu, Hengyi, et al. “Antibody conjugated magnetic iron oxide nanoparticles for cancer cell separation in fresh whole blood”, Biomaterials 32.36 (2011): 9758-9765. Others aim applications in the extraction of nucleic acids, as described in particular in the documents U.S. Pat. No. 9,976,136 or Tan, Siun Chee, and Beow Chin Yiap. “DNA, RNA, and protein extraction: the past and the present” BioMed Research International, 2009.

Applications relating to the extraction of nucleic acids generally include a plurality of successive steps including lysing the cells to release the nucleic acids, bringing the lysed solution into contact with the magnetic particles to capture the nucleic acids, applying a magnetic field (also so-called “magnetisation”) and washing. This is followed by the elution of the nucleic acids (generally by a change in pH) and the elimination of the magnetic particles. Afterwards, the isolated nucleic acids can be processed and assayed.

Nonetheless, the lysis of the cells followed by washing results in a dilution of the sample and consequently a decrease in the performances of the capture. In addition, once captured, the nucleic acids should be separated from the magnetic particles in order to be processed, which increases the number of steps.

Other applications, such as in diagnosis or oncology, aim assaying analytes directly in the blood. Thus, the prior art proposes immunoassay methods which attempt to assay proteins in a whole blood sample. The documents U.S. Pat. Nos. 6,030,845, 6,855,562, and 7,326,579, or the document of the Applicant EP2810042, are known in particular. Yet, these methods also rely on a first step of lysing the blood to destroy the cells. Lysis is followed by a step of assaying by immunoagglutination (immune-agglutination) on the lysed sample. While this lysis step allows carrying out the agglutination measurement under good conditions, it involves dilution of the relatively large sample, which has negative impact on the detection limit.

There are other immunoassay methods with magnetic particles. In particular, there are the so-called homogeneous methods and the so-called heterogeneous methods.

The homogeneous methods have the advantage of getting rid of the washing step. The documents Baudry, Jean, et al. “Acceleration of the recognition rate between grafted ligands and receptors with magnetic forces”, Proceedings of the National Academy of Sciences 103.44 (2006): 16076-16078; Ranzoni, Andrea, et al. “One-step homogeneous magnetic nanoparticle immunoassay for biomarker detection directly in blood plasma” Acs Nano 6.4 (2012): 3134-3141; and Aurich, Konstanze, et al. “Determination of the magneto-optical relaxation of magnetic nanoparticles as a homogeneous immunoassay”, Analytical chemistry 79.2 (2007): 580-586, describe homogeneous tests. Nonetheless, the absence of a washing step makes these assay methods difficult to use in complex media such as blood. Indeed, the presence of cells, in the blood in particular, considerably disturbs the measurements.

The heterogeneous methods require a larger number of steps, which makes them generally long to implement. Moreover, they are complex to implement. The document Kourilov, Vitaly, and Michael Steinitz, “Magnetic-bead enzyme-linked immunosorbent assay verifies adsorption of ligand and epitope accessibility” Analytical biochemistry 311.2 (2002): 166-170 describes a heterogeneous method.

Irrespective of the assay method, there is a risk of measuring a non-specific signal. A non-specific signal is measured in particular when at least one interfering analyte is in competition with the target analyte to bond to the magnetic particles or to the receptor specific to the target analyte.

The nature of the interfering analyte can vary. For example, it may be a protein whose three-dimensional structure is close to that of the target analyte or a molecule bearing an electric charge. More generally, an interfering analyte may be any molecule having a non-negligible affinity for the magnetic particles or for the receptors grafted at their surface. It follows that the signal measured during the assay test is never zero. Hence, the existing methods seek to reduce the probability of a non-specific signal. Nonetheless, the existing methods generally do not allow differentiating between a specific signal and a non-specific signal.

All this means that the assay methods for complex samples, and in particular total blood sample assay, are not satisfactory. The presence of cells in the sample, the need to lyse them followed by at least one washing step, and the risk of a non-specific signal could degrade the performances of the method.

The present invention improves the situation. Thus, the present invention relates to a method for assaying a target analyte in a biological sample in a liquid medium, comprising the following steps:
a. contacting the biological sample with first magnetic particles bearing a first receptor specific to a first site of attachment of the target analyte so as to form first complexes by bonding of the first magnetic particles with the target analyte this contacting being accompanied, when an interfering analyte is present in the sample, with the formation of interfering complexes by the non-specific bonding of said interfering analyte to the first magnetic particles;
b. applying a first magnetic field, and maintaining it, so as to locally combine all of the complexes formed in step a., and, if applicable, agglomerate interfering complexes with one another to form interfering aggregates;
c. negating the first magnetic field applied in step b. and adding in the liquid medium second magnetic particles bearing a second receptor specific to a second site of attachment of the analyte target;
d. measuring a first quantity representative of the amount of interfering aggregates in the liquid medium, to identify the presence or absence of said interfering 15 aggregates;
e. applying a second magnetic field so as to form second complexes by bonding the first complexes with second magnetic particles; and
f. measuring a second quantity representative of the collective amount of interfering aggregates and of second complexes in the liquid medium so as to determine the amount of second complexes formed in step e. as a function of the first quantity for deducing therefrom the amount of target analyte present in the biological sample and, if applicable, the amount of interfering analyte.

Thus, the invention allows verifying the presence or absence of an interfering analyte in the biological sample. Thus, when the biological sample includes both a target analyte and an interfering analyte, step a. comprises the formation of interfering complexes by bonding of the first magnetic particles with the interfering analyte; step b, thus comprises the formation of aggregates between the interfering complexes; step d. then predicts the quantitative measurement of the interfering aggregates formed; and step f. provides for a calculation to determine the amount of target analyte present in the biological sample, as well as the amount of the interfering analyte.

Furthermore, the method of the invention allows getting rid of an excessive dilution and consequently ensuring a good analytical sensitivity. Furthermore, the invention limits the steps in order to simplify the entire test. In particular, this results in a drastically reduced time which does not exceed the maximum duration of about 15 minutes.

Of course, when no interfering analyte is present in the biological sample, the interfering complexes, and thereby the interfering aggregates, do not form. It follows that the quantity measured in step d. is zero or does not exceed a predefined threshold value.

Thus, the method of the invention allows not only identifying the presence of an interfering analyte in the biological sample, but also quantifying the latter. In parallel, the method allows deducing the amount of target analyte in the biological sample.

Step f. may include the calculation of the difference between the second quantity measured in this step f. and the first quantity measured in step c. in order to determine the amount of target analyte.

In the main embodiment, the negation of the first magnetic field in step c. is followed or accompanied by the addition of second magnetic particles, then it is proceeded with the measurement in step d. of the first quantity representative of the amount of interfering aggregates. In another embodiment, this measurement may be carried out directly after negation of the magnetic field, i.e. before the introduction of the second magnetic particles into the liquid medium (or reaction medium).

For the measurement in step f., i.e. the measurement of the second quantity representative of the collective amount of the interfering aggregates and of the second complexes, the second magnetic field is preferably negated from said measurement beforehand. This increases the sensitivity of the measurement.

To ensure the specificity of the method of the invention, each first receiver is specific to a first site of attachment of the target analyte, and each second receiver is specific to a second site of attachment, different from the first one, of the target analyte. The flexibility resulting from two distinct sites of attachment allows adapting the method of the invention rapidly to the various needs depending on the nature of the implemented application.

In a particular embodiment of the invention which aims to extract magnetic particles from the liquid medium, the magnetic field applied in step b. is higher than or equal to 100 mT, and holding of the magnetic field lasts less than 5 minutes, and preferably less than 3 minutes. In another particular mode, the application of the first magnetic field in step b. and the application of the second magnetic field in step e. includes a magnetisation at 8 mT for 1 second, followed by three successive sequences of magnetisations and cut-offs: 15 mT for 60 seconds, 0 mT for 28 seconds, 8 mT for 1 second, 0 mT for 1 second.

This allows carrying out the method of the invention rapidly and in particular within a time period of less than 15 minutes. In addition, the selected magnetisation and cut-off sequence allows for an increased accuracy of the method of the invention by promoting the formation of the complexes between the analytes and the magnetic particles on the one hand, and by allowing for a more accurate reading on the other hand.

In a preferred embodiment, the measurements made in steps d. and f. are selected from the group including a measurement by turbidimetry, a measurement by nephelometry and a measurement by counting (by analysis and/or image processing or by flux in particular). This allows obtaining reliable results, and that being so rapidly.

In another embodiment, step a. includes the addition of a diluent so as to dilute the biological sample, the ratio between the sample and the diluent being greater than or equal to 1:10.

In particular, this dilution allows reducing the viscosity of the medium to facilitate capture of the molecule to be assayed, or in other words the formation of the first complexes. In addition, the diluent may contain agents allowing reducing the probability of aggregation of the magnetic particles together. To this end, it is possible to use non-ionic surfactants, anionic surfactants, cationic surfactants, zwitterionic surfactants, proteins (albumin, casein, gelatine, antibodies in particular) or polymers (such as polyvinylpyrrolidone, polyvinyl alcohol).

In a particular embodiment, the application of the first magnetic field in step b. allows extracting the first complexes from the liquid medium. In particular, this allows working in complex media (in whole blood, for example). Thus, a particular embodiment specifically provides for the biological sample being whole blood. An advantage of the invention is that the steps of the method can be applied directly to a blood sample, requiring no washing and/or dilution.

In another embodiment, step b. includes the elimination of the liquid medium and the addition of a reaction buffer. The reaction buffer may include an anti-aggregation agent (non-ionic surfactants, anionic surfactants, cationic surfactants, zwitterionic surfactants, proteins or polymers) and/or include a cell lysing agent (saponins, quaternary ammoniums, sodium dodecyl sulphate in particular). It is also possible to provide for at least one washing between the elimination of the liquid medium and the addition of the reaction buffer. This washing allows increasing further the specificity of the method of the invention.

The anti-aggregation agents allow dissociating any formed aggregates of magnetic particles, whereas the cell lysing agent allows lysing potential cells that would not have been evacuated with the elimination of the cell lysing agent from the liquid medium. One could note that, unlike the methods of the prior art, the lysing agent does not require any additional dilution of the sample. In this embodiment, it is then possible to provide for a more or less long incubation so as to enable a sufficient action of the agent(s) present in the reaction buffer.

Other advantages and features of the invention will become apparent upon reading the following detailed description and from the appended drawings wherein:

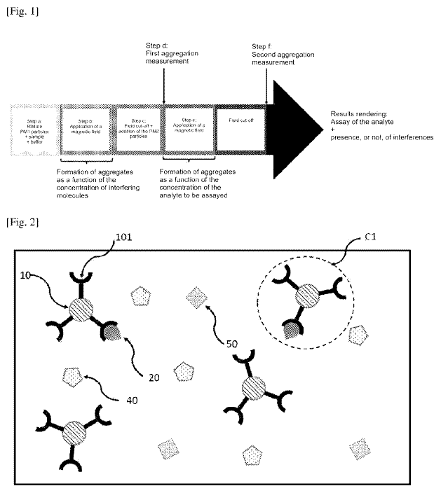

FIG. 1 shows a schematic diagram of the invention.

FIG. 2 shows a principle figure of a first step of incubation of a biological sample with first magnetic particles according to the invention;

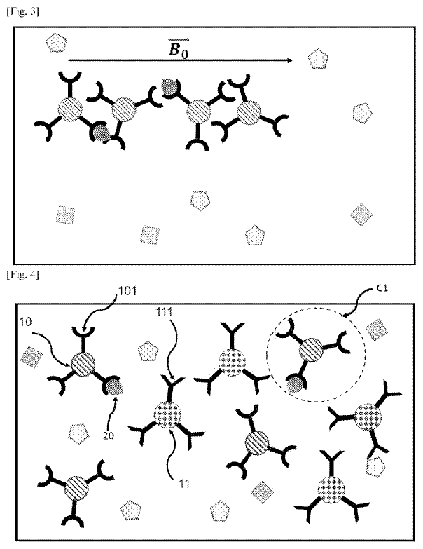

FIG. 3 shows a principle figure of a first magnetisation step of the method of the invention;

FIG. 4 shows a principle figure of a second step of incubation of a biological sample with second magnetic particles according to the invention;

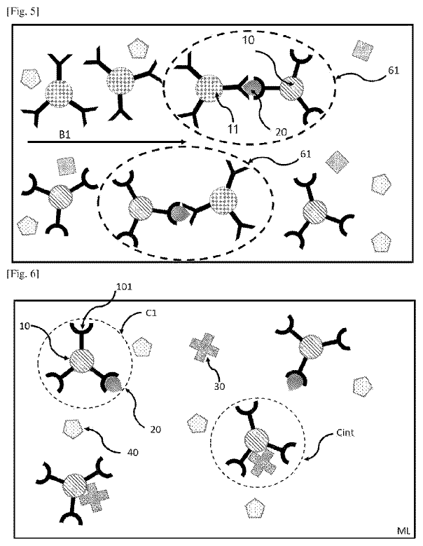

FIG. 5 shows a principle figure of a second step magnetisation of the method of the invention;

FIG. 6 shows a principle figure of a first step of incubation of a biological sample including interfering molecules with first magnetic particles according to the invention;

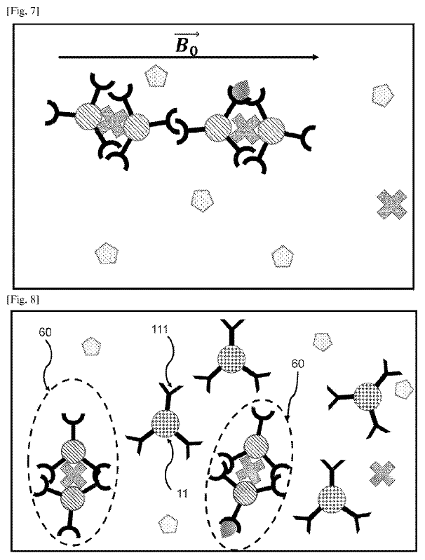

FIG. 7 shows a principle figure of a first step of magnetisation of the method of the invention applied to a biological sample including interfering molecules;

FIG. 8 shows a principle figure of a second step of incubation of a biological sample including interfering molecules with second magnetic particles according to the invention;

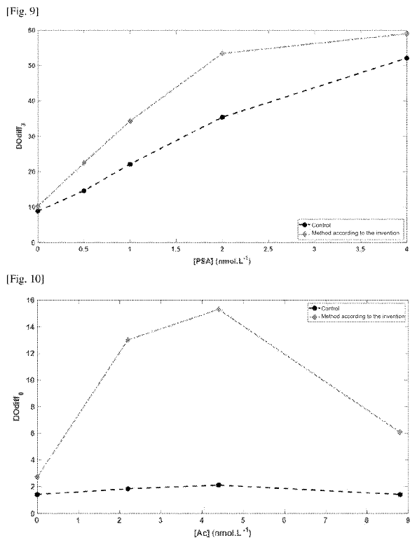

FIG. 9 shows a graph of the optical density as a function of a concentration of the prostate specific antigen (PSA) in a first embodiment;

FIG. 10 shows a graph of the optical density as a function of an antibody concentration (Ac) of a second embodiment;

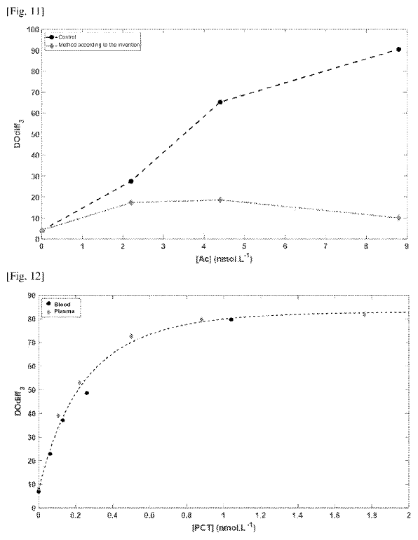

FIG. 11 shows a graph of the optical density as a function of an antibody concentration (Ac) of the second embodiment;

FIG. 12 shows a graph of the optical density as a function of a concentration of the anti-PCT (procalcitonin) antibodies of a third embodiment;

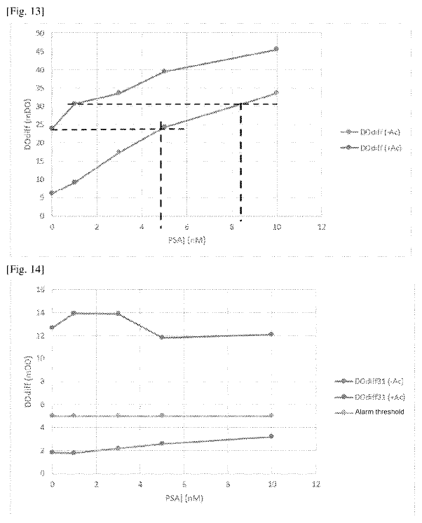

FIG. 13 shows a graph of the optical density as a function of a concentration of the prostate specific antigen (PSA) of a sixth example (not according to the invention); and

FIG. 14 shows a graph of the optical density as a function of a concentration of the prostate specific antigen (PSA) of a seventh example (according to the invention).

The figures, tables and the description hereinafter essentially contain elements of certain nature. The figures and tables are an integral part of the description, and will therefore not only be used to better understand the present invention, but also contribute to the definition thereof, where appropriate.

In general, the present invention relates to the identification of an analyte in a liquid biological sample. The sample may be of any suitable type, such as for example a bone marrow sample, cerebrospinal fluid, lymph, urine, or preferably a whole blood sample. For example, the target analyte may be a protein, a nucleic acid or any other molecule of interest present in the sample. The target analyte is present in a substantially large amount in the sample. Thus, a sample may comprise a target molecule present in a substantially large number of copies. In other words, the concentration of target analyte may be substantially high in the sample. This also applies to any other non-target analyte potentially present in the sample.

The invention uses a technique for capturing the target analyte(s) by magnetic particles that have been functionalised at the surface with ligands specific to each target analyte. These may consist of particles of the above-described type. In general, the diameter of the magnetic particles used is comprised between 5 nm and 10,000 nm, preferably between 100 nm and 500 nm.

In the assay method of the invention, a first step a. includes contacting the biological sample with first magnetic particles

FIG. 1 shows a schematic diagram of the invention.

Thus, this diagram generally shows an embodiment of the implementation of the method of the invention. In a first step a., first magnetic particles are mixed with a liquid biological sample in which there is, or not, a target analyte to be assayed in the presence of a buffer. The biological sample may further, yet not necessarily, include an interfering analyte.

First, the biological sample is brought into contact with first magnetic particles. Each magnetic particle bears a receptor specific to a first site of attachment of the target analyte so as to form first complexes by bonding first magnetic particles with the target analyte when the latter is in the biological sample. When an interfering analyte is present in the sample, this contacting is accompanied with the formation of interfering complexes by the non-specific bonding of the interfering analyte with the first magnetic particles. Logically, when there is no interfering analyte in the biological sample, no interfering complexes are formed.

FIG. 2 shows a biological sample including a liquid medium containing molecules. [FIG. 2] shows first magnetic particles 10 or PM1 bearing specific receivers, herein so-called first receivers 101. These first magnetic particles 10 are brought into contact with the molecule to be assayed present in the biological sample. The molecule to be assayed is referred to as the target analyte 20. The biological sample further includes other species or molecules 40, 50 which may for example be soluble molecules, cells or particles (or interfering molecules 30 as will be seen later on).

A consequence of this contacting of step a. is that the molecule 20 is captured by the first receivers 101. For this purpose, the first receivers 101 are specific to the target analyte 20. For good specificity, monoclonal antibodies or parts of antibodies are used as receivers. Bonding between each receiver and each target analyte results in the formation of complexes, which are herein referred to as the first complexes C1. Hence, the complexes are formed by the first magnetic particles 10 associated with the target analytes 20. These complexes are dispersed in the liquid medium.

In general, the contacting of step a. may last for about 10 minutes, preferably less than 5 minutes. Optionally, it is possible to provide for mixing or stirring the liquid medium during the incubation phase in order to increase the capture efficiency.

Step a. may include the addition of a diluent so as to dilute the biological sample, or more particularly the liquid medium. In general, we will select a dilution that does not exceed a division of the initial concentration by ten (i.e. 10×).

Afterwards, the method of the invention provides for a step b., in which a first magnetic field is applied. This allows locally combining all of the complexes formed in step a., and when present agglomerating interfering complexes together in order to form interfering aggregates. By locally combining the complexes, it should be understood an organisation of the latter within the liquid medium. Depending on the magnetic field applied, the location may vary.

One should retain that a main effect of this step b., and more particularly of the application of the magnetic field, is actually the formation of interfering aggregates originating from the agglomeration of interfering complexes with one another, when these have formed in step a. of bringing the biological sample into contact with the first magnetic particles.

In a particular embodiment of the invention, this step provides for attracting the first magnetic particles to a magnet. Consequently, the first complexes formed in step a. which essentially consist of the association of the first magnetic particles with the target analyte, are attracted to the magnet. As a result, all of the complexes formed in step a. are combined locally against the magnet, or against an environment close to the magnet. This allows isolating the first complexes and separating them from the liquid medium. In other words, in this particular embodiment, the first complexes of the liquid medium of the biological sample are extracted. Logically, the separation of the first complexes from the liquid medium will be complete only when the liquid medium is evacuated later on in the process. In this embodiment, step b. may provide for maintaining the magnetic field for some time, for example 5 to 10 minutes, preferably less than 5 minutes. This contributes to the good extraction of all of the first complexes formed in step a. of the liquid medium.

FIG. 3 shows the magnetic particles 10 attracted or organised according to the field B₀ generated by a magnet. The first complexes C1 are assembled locally.

In a particular embodiment, the elimination of the liquid medium from the biological sample is provided, then the addition of a reaction buffer. In other words, the liquid medium is replaced by a reaction buffer, which is also liquid. For this purpose, the liquid medium of the reaction vessel is first evacuated, then the reaction buffer is poured into the vessel. In this embodiment, a permanent magnet is generally provided which allows assembling the first complexes C1 bearing the target analyte 20 locally against the latter. The target analyte molecules 20, bonded to the receptors 101 are isolated and preserved during the evacuation of the liquid medium from the biological sample. The reaction buffer is added as a replacement for the liquid medium.

In this embodiment, one or more washes may be provided between the elimination of the liquid medium and the addition of the reaction buffer. This washing allows increasing the specificity of the method of the invention. During the washing operation(s), the magnetic field may be maintained or cut off. In the latter case, it is necessary to re-apply the magnetic field for a given time period after each washing so as to capture the suspended magnetic particles again.

In one embodiment, the reaction buffer may comprise one or more anti-aggregation agent(s) and/or one or more cell lysing agents. The anti-aggregation agents allow dissociating any aggregates or clusters of magnetic particles formed in step a. and/or in step b. Thus, the accuracy of the method of the invention is increased. The cell lysing agent allows lysing any cells that would have not been discharged with the elimination of the above-described liquid medium. One could note that, unlike the methods of the prior art, the lysis agent does not require an additional dilution of the sample.

Step c. of the method of the invention includes negating the magnetic field B₀. When the field is cut off, the particles and complexes, as well as any formed aggregates, scatter in the liquid medium.

FIG. 4 shows the complexes C1 resuspended. Step c. further includes the addition in the liquid medium of second magnetic particles 11, or PM2, carrying a second receiver 111 specific to the target analyte 20. At this stage, the second receivers 111 of the second magnetic particles 11 do not bond, or very little, to the target analyte 20. Where necessary, it is possible to provide for stirring the reaction medium.

It should be noted that the target analytes 20 generally have a three-dimensional shape allowing selecting diversified specific receivers 101, 111. Thus, each first receiver 101 is specific to a first attachment site of the target analyte 20 and each second receiver 111 is specific to a second attachment site of the target analyte, different from the first one. This allows increasing the specificity of the method of the invention. This flexibility originating from two attachment sites also allows rapidly adapting the method of the invention to the various needs, and in particular according to the nature of the implemented application. Thus, it is possible to diversify the receptors according to the target analyte to be assayed.

Afterwards, the method of the invention provides for a step d., in which a first quantity representative of the amount of interfering aggregates in the liquid medium is measured, to identify the presence or absence of said interfering aggregates. When the measured quantity is zero or is below a predefined threshold value, there is no (or extremely few) interfering aggregates in the liquid medium. In this case, it is possible to consider the principle that there is no (or extremely few) interfering analytes in the biological sample. When the measured quantity is non-zero or exceeds the predefined threshold value, there are interfering aggregates in the liquid medium, and therefore interfering analyte in the biological sample.

In particular, the measurement may be carried out by turbidimetry, by nephelometry or by counting (by analysis and/or image processing or by flux in particular).

Afterwards, the method of the invention provides for a step e., in which a second magnetic field is applied so as to form second complexes by bonding of the first complexes C1 to the second magnetic particles 11, or more specifically to the receivers 111 borne by the second magnetic particles. In terms of detectability, these second complexes are comparable to the interfering aggregates, i.e. it is relatively easy to detect them by optical density measurements in particular.

FIG. 5 shows the second complexes 61 generated by the application of the second magnetic field B1. The target analyte 20 is sandwiched by the first and second receivers respectively borne by the first 10 and second 11 magnetic particles. In general, this step e. aims to generate links or clusters of magnetic particles. The “sandwich” complexes or molecules, formed by the first 10 and second 11 magnetic particles bordering the target analyte 20, are detectable by different techniques including in particular an optical density measurement.

The documents WO 2009/034271, FR2919390 and FR2959820 describe assay methods in which a series of cycles of applications and cut-offs of a magnetic field are applied to the liquid reaction medium to cause the formation of links or clusters of magnetic particles during the reaction between the magnetic particles and a target analyte. Each application of the field results in an increase in the number of analyte/magnetic particle bonds and each cut-off of the field causes a dispersion of the unbound magnetic particles in the liquid medium. Throughout the process, the number and/or size of the links or clusters of bound particles increase. This proportionally increases the turbidity of the reaction medium. Thus, the measurements of the optical density of the medium after each cut-off of the magnetic field allow calculating the concentration of the target analyte in the sample.

Other types of magnetic pulse sequences are possible. Examples are described in the publication Fast Magnetic Field-Enhanced Linear Colloidal Agglutination Immunoassay, Daynes et al. Anal. Chem. 2015, 87, 7583-7587.

In the present invention, the first magnetic field B₀ applied during step b. may be identical or different from the second magnetic field B1 applied in step e.

It may be advantageous to apply magnetic fields having different properties during these two steps. This is the case in the embodiment where the extraction of the first complexes of the liquid medium is provided for. Under these conditions, to enables an optimum capture of the first magnetic particles 10, the field B₀ of step b. must combine a significant intensity (typically higher than 100 mT) with a significant gradient in the direction of magnetisation. On the contrary, it is preferable to apply a field B1 with a moderate intensity (typically lower than 50 mT) and the gradient of which is low for step e. The field B1 of step e. may also be applied in the form of several pulses, separated by relief periods. The duration and the intensity of these pulses may be variable.

More generally, the application of the second magnetic field B1 preferably includes a plurality of magnetic pulses, separated by rest times. In one embodiment, this step e. provides for a magnetisation at 8 mT for 1 second, followed by the following three successive sequences of magnetisations and cut-offs: 15 mT for 60 seconds, 0 mT for 28 seconds, 8 mT for 1 second, 0 mT for 1 second:
1 s, 8 mT+3×[60 s, 15 mT+28 s, 0 mT+1 s, 8 mT+1 s, 0 mT].

The different pulses at 8 mT for 1 second essentially allow for a more accurate reading of the state of aggregation of the PM1 particles in case of presence of an interfering analyte. The series of pulses/rests of 15 mT for 60 seconds, followed by rest (0 mT) for 28 seconds, followed by 8 mT for 1 second, followed by rest (0 mT) for 1 second essentially enables the formation of complexes.

The second complexes 61 form aggregates which are detectable by an optical density measurement or by counting for example. More generally, the second complexes can be detected because the magnetic particles are aggregated together because of bonding of the target analyte to the two receptors (sandwich), while the signal of a first complex does not significantly differ from that of a free magnetic particle, given the difference in size (and/or optical index and/or magnetic moment) between a magnetic particle and a target analyte molecule.

Thus, step f. of the method of the invention aims to determine the amount of second complexes formed in order to deduce therefrom the amount of target analytes present in the biological sample. For this purpose, step f. provides for measuring all aggregates, i.e. all of the second complexes and, when present, interfering aggregates. In fine, this measurement allows determining the amount of second complexes 61 formed in step e., and this in particular as a function of the measurement of interfering aggregates alone performed beforehand in step d. Afterwards, the result of the measurement allows calculating the amount of target analyte 20 present in the biological sample.

The invention has the advantage of identifying not only the target analyte, but also an interfering analyte, or several interfering analytes.

Reference is now made to FIGS. 6, 7 and 8 to describe in more detail the steps of the method with the presence of an interfering analyte.

Step a. (cf. [FIG. 6]) in this case comprises not only the formation of the first complexes C1, but also the formation of interfering complexes Cint by bonding between the first magnetic particles 10 and an interfering analyte 30. The interfering complexes Cint are then dispersed in the liquid medium of the biological sample.

More generally, in step a, the magnetic particles 10 bearing specific receptors 101 are brought into contact with the molecule to be assayed 20, in the presence of other species 30, 40, which may be soluble molecules, cells, particles, or the like. One of these species (molecules 30 in [FIG. 6]) bonds in a non-specific manner to the first magnetic particles 10. Nevertheless, the molecule 20 is captured by the receivers 101.

During step b. (cf. [FIG. 7]), the first magnetic particles 10 are attracted or organised locally within the liquid medium according to the field B₀ generated by the magnet. In the particular embodiment of the invention described hereinabove with reference to the extraction of the magnetic particles, the liquid medium is replaced by the reaction buffer. In this specific case, a permanent magnet (not shown in the drawings), which attracts the magnetic particles, should be used.

More generally, the molecules 20 (target analyte) bonded to the receptors 101 as well as the molecules 30 bonded in a non-specific manner to the magnetic particles 10 are locally combined within the liquid medium. This local assembly at a given location within the liquid medium of the first complexes C1 and of the interfering complexes Cint results in the formation of non-specific aggregates 60 (or interfering aggregates 60) between the first complexes C1 and the interfering complexes Cint. These non-specific aggregates may also be formed by the agglomeration of the interfering complexes Cint together. More generally, the non-specific aggregates form essentially identically to the complexes of interest, i.e. between a complex Cint and a neighbouring magnetic particle. The latter may be bonded to an analyte molecule (C1), to an interfering molecule (Cint), or be a free particle PM1. These non-specific aggregates 60 are detectable in a liquid medium, by turbidimetry measurement, for example (cf. [FIG. 8]).

Once the aggregates are formed, step d. enables the quantitative measurement thereof in the liquid medium.

As explained hereinabove, step f. uses the measurement made during step d. to calculate the amount of the specific aggregates formed by the second complexes. To this end, the calculation takes account of both the measurement made in step d. and that made in step f. Thus, it is possible to deduce therefrom the amount of target analyte present in the biological sample.

The measurement of the aggregation state during step d. allows limiting the risk of interference that might lead to false positive results. In the presence of a molecule that might lead to such interferences, step b. may result in the formation of non-specific aggregates of first magnetic particles 10 (PM1). In the absence of step d., these aggregates would be detected only during step f., without it being possible to distinguish the specific aggregates, caused by the presence of the molecule to be assayed (the target analyte), the non-specific aggregates, caused by the presence of another molecule (the interfering analyte). By applying step d., it is for example possible to determine an initial aggregation threshold beyond which no result will be rendered, due to interference, thereby avoiding erroneous results. One advantage of the invention is that the aggregation measurements during steps d. and f. could be combined to calculate an estimate of the specific aggregation, excluding the non-specific aggregation.

Optionally, it is also possible to stop the method of the invention after step d. If the measured amount of non-specific aggregates is too high and/or exceeds a predefined sensitivity threshold, the method of the invention may be interrupted after making the measurement of step d. It is then possible to restart the method of the invention by selecting other receivers specific to the target analyte.

In a particular embodiment of the invention, the above-described method is therefore stopped after step d., namely after measurement of the first amount representative of the amount of interfering aggregates in the liquid medium. To this end, it is possible to set a threshold quantity beyond which the results are not sensitive enough. Thus, the execution of the subsequent steps which become useless or at least unusable is avoided. Thus, unnecessary costs and time losses can be avoided.

### EMBODIMENTS

A first series of experiments (examples 1 to 3) is carried out in order to demonstrate the feasibility and sensitivity of the invention.

### Example 1

A PSA (prostate-specific antigen) assay test is implemented. Magnetic particles (Carboxyl Adembeads 200 nm, Ademtech), are functionalised with anti-PSA antibodies. A batch of particles is functionalised with a first clone (P4, reference 7820-0370, available from the company Bio-Rad). With reference to the general description, these are the first magnetic particles PM1. A second batch is functionalised with a second clone recognising a different epitope (214, reference 7820-0217, available from the company Bio-Rad). With reference to the general description, these are the second magnetic particles PM2. The samples consist of PSA (reference P3338, available from the company Sigma-Aldrich) purified at 10 nM diluted in horse serum (reference H1270, available from the company Sigma-Aldrich) at different concentrations. Hence, the target PSA concentration in the samples is known.

The assay of the samples is carried out as follows:
71.5 μL of the sample are brought into contact with 1.5 μL of PM1 (P4) functionalised particles at 1% for 2 min;
The medium is magnetised for 3 min on a permanent magnet, and the supernatant is eliminated;
the particles are supplemented with 73.5 μL of HEPES buffer 50 mM, pH 7.5; F108 0.8%; NaCl 800 mM; 0.09% NaN₃ and 1.5 μL of PM2 (214) functionalised particles at 1% are added.

The reaction medium thus formed is subjected to the following magnetic field sequence:
1 s, 8 mT+3×[60 s, 15 mT+28 s, 0 mT+1 s, 8 mT+1 s, 0 mT],

The light intensity through the reaction medium illuminated by an RC-LED at 650 nm is measured and the difference in optical density before and after application of the last pulse with an 8 mT intensity (Dodiff₃) is used as signal of interest.

Control measurements are made with the same samples and the same particles, but without an extraction step (step c. with reference to the general description). The reaction medium, consisting of 57 μL of HEPES buffer 50 mM, pH 7.5; F108 0.8%; NaCl 800 mM; NaN3 0.09%, 15 μL of the sample and 1.5 L of each batch of functionalised particles, is subjected to the same magnetic field cycle as described hereinabove.

the results which appear in Table 1 hereinafter, as a function of the PSA concentration in the samples tested, are obtained.

TABLE 1

| | | | | | |
|---|---|---|---|---|---|
| [PSA] (nM) | 0 | 0.5 | 1 | 2 | 4 |
| Dodiff3 | 10.27 | 22.49 | 34.35 | 53.45 | 59.01 |
| (extraction) | | | | | |
| Dodiff3 | 8.87 | 14.62 | 22.14 | 35.42 | 52.12 |
| (control) | | | | | |

FIG. 9 shows the results and the corresponding curves of Dodiff3 as a function of the PSA concentration.

One could notice that the method using the method according to the invention allows obtaining a signal increasing with the PSA concentration in the sample, and with an activity higher than the control tests.

### Example 2

Tests allowing simulating the presence of an interfering molecule in the sample of interest are implemented. Magnetic particles (Carboxyl Adembeads 200 nm, from Ademtech), are functionalised with anti-PSA antibodies. A batch of PM1 particles is functionalised with a first clone (P4, reference 7820-0370, available from the company Bio-Rad), and a second batch PM2 is functionalised with a second clone recognising a different epitope (214, reference 7820-0217, available from the company Bio-Rad). The samples consist of a 9 g/l NaCl solution in which an anti-mouse IgG antibody (Ac) is added (M8642, available from the company Sigma-Aldrich) capable of bonding in a non-specific manner to the anti-PSA antibodies grafted onto the particles.

The assay of the samples is carried out as follows:

71.5 μL of the sample are brought into contact with 1.5 L of the PM1 (P4) functionalised particles at 1% for 2 min;

The medium is magnetised for 3 min on a permanent magnet, and the supernatant is eliminated;

The particles are supplemented with 73.5 μL of HEPES buffer 50 mM, pH 7.5; F108 0.8%; NaCl 800 mM; NaN3 0.09% and 1.5 μL of PM2 (214) functionalised particles at 1% are added.

The reaction medium thus formed is subjected to the following magnetic field sequence:
1 s, 8 mT+3×[60 s, 15 mT+28 s, 0 mT+1 s, 8 mT+1 s, 0 mT],

The light intensity through the reaction medium illuminated by an RC-LED at 650 nm is measured and the difference in optical density before and after application of the last pulse with an 8 mT intensity (Dodiff3) is used as a signal of interest. This same signal may be measured during the application of the first pulse with an 8 mT intensity (Dodiff0) to determine the aggregation of the medium before application of the magnetic field.

Control measurements are made with the same samples and the same particles, but without an extraction step. The reaction medium, consisting of 57 μL of HEPES buffer 50 mM, pH 7.5; F108 0.8%; NaCl 800 mM; NaN3 0. 09%, 15 μL of the sample and 1.5 μL of each batch of functionalised particles, is subjected to the same magnetic field cycle as described before.

The results which appear in Table hereinafter, as a function of the concentration of antibodies in the reaction media, with respect to the measurement before agglutination, are obtained:

TABLE 2

| | | | | |
|---|---|---|---|---|
| [Ac] ( nM) | 0 | 2.2 | 4.4 | 8.8 |
| Dodiff₀ | 2.72 | 13.02 | 15.3 | 6.08 |
| (extraction) | | | | |
| Dodiff₀ | 1.41 | 1.84 | 2.12 | 1.41 |
| (control) | | | | |

FIG. 10 shows the results and the corresponding curves of Dodiff3 as a function of the Ac concentration before aggregation.

While the signal before application of the field does not depend on the concentration of antibodies interfering with the control measurement, one could notice that the addition of this antibody results in an increase in the aggregation signal before application of the field because of the magnetisation step, which allows considering alarming these measurements.

The following results which appear in Table 3 below, as a function of the concentration of antibodies in the reaction media, with regards to the measurement after agglutination, are obtained:

TABLE 3

| | | | | |
|---|---|---|---|---|
| [Ac] (nM) | 0 | 2.2 | 4.4 | 8.8 |
| Dodiff₃ | 4.1 | 17.29 | 18.57 | 10.01 |
| (extraction) | | | | |
| Dodiff₃ | 3.95 | 27.47 | 65.23 | 90.46 |
| (control) | | | | |

FIG. 11 shows the results and the corresponding curves of Dodiff3 as a function of the Ac concentration after aggregation.

In both cases, the aggregation signal is increased in the presence of the interfering antibody. Thus, in the absence of the initial measurement made during the extraction, it would be impossible to determine whether the sample contains PSA or an interfering molecule.

### Example 3

Tests allowing verifying the applicability of the method of the invention on a whole blood sample are carried out.

Magnetic particles (Carboxyl Adembeads 200 nm, available from the company Ademtech), are functionalised with anti-PCT (procalcitonin) antibodies. A batch of PM1 particles is functionalised with a first clone (E86813M, available from the company Meridian Life Sciences), and a second batch PM2 is functionalised with a second clone recognising a different epitope (E01342M, available from the company Meridian Life Sciences). The samples consist of recombinant PCT at 400 nM diluted in human blood at different concentrations. Hence, the target PCT concentration in the samples is known.

The assay of the samples is carried out as follows:
71.5 μL of the sample are brought into contact with 1.5 μL of functionalised particles E86813M at 1% and 71.5 μL of HEPES buffer 50 mM, pH 7.5; F108 0.8%; NaCl 800 mM; DTT 10 mM; NaN3 0.09% for 2 min;
The medium is magnetised for 3 min on a permanent magnet, and the supernatant is eliminated;
The particles are supplemented with 73.5 μL of HEPES buffer 50 mM, pH 7.5; F108 0.8%; NaCl 800 mM; DTT 10 mM; NaN3 0.09% and 1.5 μL of functionalised particles E01342M at 1% are added.

The reaction medium thus formed is subjected to the following magnetic field sequence:
1 s, 8 mT+3×[60 s, 15 mT+28 s, 0 mT+1 s, 8 mT+1 s, 0 mT].

The light intensity through the reaction medium illuminated by an RC-LED at 650 nm is measured and the difference in optical density before and after application of the last pulse of 8 mT intensity (Dodiff3) is used as a signal of interest.

The same tests are carried out on the plasma samples derived from the blood samples. These are centrifuged for 10 min at 1,500 g in order to extract the plasma therefrom. Since the haematocrit of the blood sample is 40.4%, it is then possible to calculate the expected plasma concentration of PCT as a function of the expected concentration of PCT in the blood.

The results which appear in the following tables 4 (blood) and 5 (plasma) are obtained:

TABLE 4

| | | | | | |
|---|---|---|---|---|---|
| [PCT]_{blood} (nM) | 0 | 0.0625 | 0.13 | 0.26 | 1.04 |
| Dodiff₃ | 6.83 | 22.82 | 37.1 | 48.62 | 79.75 |

TABLE 5

| [PCT]ₚₗₐₛₘₐ (nM) | 0 | 0.106 | 0.22 | 0.5 | 0.88 | 1.76 |
|---|---|---|---|---|---|---|
| Dodiff₃ | 8.19 | 39.02 | 52.99 | 72.81 | 79.74 | 81.91 |

FIG. 12 shows these results in a graph.

One could notice that the signal measured as a function of the PCT concentration in the sample is independent of the nature of the latter, whether blood or plasma. This demonstrates the possibility of applying the described method to a whole blood sample.

A second series of experiments (examples 4 to 7) is carried out in order to demonstrate the effect of an interfering antibody with the aggregation of magnetic particles.

For this purpose, aggregation measurements in the presence of different concentrations of anti-mouse antibodies are carried out. This is done to simulate or mimic the effect of an interfering antibody.

For this series of experiments, we use the particles Adembeads 200 nm functionalised either by the antibody P4 (reference 7820-0370, available from the company Bio-Rad), or by the antibody 214 (reference 7820-0217, available from the company Bio-Rad).

### Example 4: PSA-Free Measurements

In this example, the first and second magnetic particles (PM1 and PM2) are introduced into the liquid medium simultaneously. Consequently, it is not an example according to the invention.

In a first step, a standard cycle is carried out. The prepared samples comprise:
57 μL of HEPES buffer 50 mM, pH 7.5; F108 0.8%; NaCl 800 mM;
15 μL of anti-mouse antibodies (M8642, Sigma-Aldrich) at the desired concentration in a 9 g/l NaCl solution;
3 μL of P4 particles 1% w/v (weight per volume, or p/v weight per unit volume); and
3 μL of 214 particles 1% w/v

Afterwards, the following cycle (Cagg) is applied to the samples:

1 s, 8 mT+3×[60 s, 15 mT+28 s, 0 mT+1 s, 8 mT+1 s, 0 mT].

It is possible to measure the difference in optical density for each sample before and after the first pulse of intensity 8 mT (Dodiff0) as an indicator of the initial level of aggregation of the particles. To measure the final aggregation level, it is possible to measure the difference in optical density before and after the last pulse of intensity 8 mT (Dodiff3) or the difference in optical density between the end and the start of the Cagg cycle (ΔDO).

The results which appear in Table 6, as a function of the concentration of antibody (Ac) in the reaction medium, are obtained:

TABLE 6

| [Ac] (nM) | 0 | 1 | 2 | 3 | 4 | 5 |
|---|---|---|---|---|---|---|
| ΔDO (mDO) | 2.86 | 25.62 | 51.46 | 74.37 | 79.03 | 101.08 |
| Dodiff₀ (mDO) | 2.71 | 2.23 | 2.58 | 2.28 | 2.58 | 2.78 |
| Dodiff₃ (mDO) | 6.08 | 23.89 | 42.62 | 56.59 | 60.09 | 71.79 |

One could notice that the selected antibody causes an interference since Dodiff3 or ΔDO increase with the concentration of antibody introduced. In turn, the measurement of Dodiff0 does not vary significantly with the concentration of antibody, which therefore does not allow alarming this interference.

### Example 5: PSA-Free Measurements

The prepared samples comprise:
57 μL of HEPES buffer 50 mM, pH 7.5; F108 0.8%; NaCl 800 mM;
15 μL of anti-mouse antibodies (M8642, Sigma-Aldrich) at the desired concentration in a 9 g/l NaCl solution; and
3 μL of P4 particles 1% w/v

The Cagg cycle (detailed hereinabove) is applied to this sample, then 3 μL of 214 particles 1% w/v are added to the medium and the Cagg cycle is applied again.

It is possible to measure the same indicators as before on the first cycle, applied only to the P4 particles (Dodiff₀¹, Dodiff31, ΔDO¹), or on the second cycle, applied to all of the particles (Dodiff₀², Dodiff₃², ΔDO²). The results which appear in Table 7 hereinafter are obtained:

TABLE 7

| [Ac] (nM) | 0 | 1 | 2 | 3 | 4 | 5 |
|---|---|---|---|---|---|---|
| ΔDO¹ (mDO) | 1.39 | 13.96 | 25.84 | 31.87 | 35.36 | 45.75 |
| Dodiff₀¹ (mDO) | 1 | 0.99 | 1.09 | 0.89 | 0.77 | 0.96 |
| Dodiff₃¹ (mDO) | 1.82 | 12.7 | 22.15 | 25.67 | 28.19 | 34.69 |
| ΔDO² (mDO) | 2.22 | 19.65 | 40.27 | 50.02 | 54.94 | 61.28 |
| Dodiff₀² (mDO) | 3.21 | 13.72 | 22.83 | 26.53 | 28.59 | 34.63 |
| Dodiff₃² (mDO) | 4.87 | 28.54 | 51.43 | 56.41 | 63.23 | 74.97 |

The indicators ΔDO² or Dodiff32 increase with the concentration of antibody introduced. However, with this protocol, it is possible to use the values of ΔDO¹ or Dodiff₃¹ to detect the presence of aggregates prior to the application of the second cycle, related to an interference in the sample. Thus, in this example, the application of a threshold selected at 5 mDO allows detecting the samples containing the interfering antibody.

### Example 6: PSA Dose-Response Curves

In this example, the first and second magnetic particles (PM1 and PM2) are introduced into the liquid medium simultaneously. Consequently, this is not an example according to the invention.

First, an interfering antibody-free dose-response curve is plotted.

The samples comprise:
57 μL of HEPES buffer 50 mM, pH 7.5; F108 0.8%; NaCl 800 mM;
15 μL of PSA (P3338, Sigma-Aldrich) at the desired concentration in a 9 g/l NaCl solution;
3 μL of P4 particles 1% w/v; and
3 μL of 214 particles 1% w/v

After application of the Cagg cycle, the results which appear in Table 8 hereinafter are obtained:

TABLE 8

| | | | | | |
|---|---|---|---|---|---|
| [ PSA] (nM) | 0 | 1 | 3 | 5 | 10 |
| ΔDO (mDO) | 2.86 | 6.45 | 14.27 | 20.83 | 33.28 |
| Dodiff₀ (mDO) | 2.71 | 2.23 | 2.18 | 2.56 | 2.11 |
| Dodiff₃ (mDO) | 6.08 | 9.11 | 17.32 | 24.24 | 33.5 |

Afterwards, a dose-response curve by adding an anti-mouse antibody is plotted.

For this purpose, the samples comprise:
57 μL of HEPES buffer 50 mM, pH 7.5; F108 0.8%; NaCl 800 mM;
15 μL of PSA (P3338, Sigma-Aldrich) at the desired concentration in a 9 g/l +5 nM NaCl solution of anti-mouse antibodies;
3 μL of P4 particles 1% w/v; and
3 μL of 214 particles 1% w/v.

Hence, there is an antibody concentration of 1 nM in the reaction medium. After application of the Cagg cycle, the results which appear in Table 9 hereinafter are obtained:

TABLE 9

| | | | | | |
|---|---|---|---|---|---|
| [PSA] (nM) | 0 | 1 | 3 | 5 | 10 |
| ΔDO (mDO) | 25.62 | 30.98 | 38.59 | 42.72 | 49.9 |
| Dodiff₀ (mDO) | 2.23 | 2.48 | 2.17 | 2.35 | 2.07 |
| Dodiff₃ (mDO) | 23.89 | 30.63 | 33.57 | 39.42 | 45.52 |

FIG. 13 reports these results in a graph.

One could notice that in the absence or in the presence of anti-mouse antibodies, the value of Dodiff₀ does not vary significantly, and therefore does not allow predicting the presence of an interfering molecule. Yet, this molecule leads to an overestimation of the values of Dodiff3 or of ΔDO. Thus, by reporting the ΔDO values measured in the presence of the antibody on the dose-response curve in the absence of antibodies, used a as standard curve, this results in an overestimation of the PSA titres. For example, the sample including no PSA would be assayed at about 5 nM, and the sample containing 1 nM of PSA would be titrated at more than 8 nM.

### Example 7: PSA Dose-Response Curves

First, an interfering antibody-free dose-response curve is plotted.

The samples comprise:
57 μL of HEPES buffer 50 mM, pH 7.5; F108 0.8%; NaCl 800 mM;
15 μL of PSA (M8642, Sigma-Aldrich) at the desired concentration in a 9 g/l NaCl solution; and
3 μL of P4 particles 1% w/v.

The Cagg cycle is applied to this sample, then 3 μL of 214 particles 1% w/v are added to the medium and the Cagg cycle is applied again.

The results which appear in Table 10 hereinafter, as a function of the amount of PSA present in the reaction medium, are obtained:

TABLE 10

| | | | | | |
|---|---|---|---|---|---|
| [Ac] (nM) | 0 | 1 | 3 | 5 | 10 |
| ΔDO¹ (mDO) | 1.39 | 0.93 | 1.07 | 1.23 | 1.89 |
| Dodiff₀¹ (mDO) | 1 | 0.72 | 0.86 | 0.9 | 0.77 |
| Dodiff₃¹ (mDO) | 1.82 | 1.78 | 2.19 | 2.58 | 3.21 |
| ΔDO² (mDO) | 2.22 | 11.51 | 22.98 | 33.79 | 51.34 |
| Dodiff₀² (mDO) | 3.21 | 2.98 | 3.6 | 3.44 | 4.57 |
| Dodiff₃² (mDO) | 4.87 | 14.03 | 25.83 | 34.35 | 47.64 |

One could notice that while the values of Dodiff₃ or of ΔDO increases with the amount of PSA after the second agglutination cycle, as expected, they do not vary significantly after the first agglutination cycle, since the aggregates form only between particles bearing the antibody P4 and particles bearing the antibody 214. Hence, these samples will not be alarmed while keeping the assumption of a threshold at 5 mDO as described hereinabove.

Afterwards, a dose-response curve is plotted by adding an anti-mouse antibody.

The samples comprise:
57 μL of HEPES buffer 50 mM, pH 7.5; F108 0.8%; NaCl 800 mM;
15 μL of PSA (M8642, Sigma-Aldrich) at the desired concentration in an NaCl solution 9 g/l+5 nM of anti-mouse antibodies; and
3 μL of P4 particles 1% w/v

The Cagg cycle is applied to this sample, then 3 μL of 214 particles 1% w/v are added to the medium and the Cagg cycle is applied again.

the results which appear in Table 11 hereinafter, as a function of the amount of PSA present in the reaction medium, are obtained:

TABLE 11

| | | | | | |
|---|---|---|---|---|---|
| [Ac] (nM) | 0 | 1 | 3 | 5 | 10 |
| ΔDO¹ (mDO) | 13.96 | 14.92 | 13.9 | 11.83 | 11.63 |
| Dodiff₀¹ (mDO) | 0.99 | 0.89 | 0.87 | 0.8 | 0.72 |
| Dodiff₃¹ (mDO) | 12.7 | 13.95 | 13.88 | 11.82 | 12.09 |
| ΔDO2 (mDO) | 19.65 | 28.29 | 40.83 | 48.06 | 58.91 |
| Dodiff₀² (mDO) | 13.72 | 15 | 14.69 | 12.69 | 13.06 |
| Dodiff₃² (mDO) | 28.54 | 36.01 | 44.95 | 47.54 | 56.29 |

The values of Dodiff32 or of ΔDO² are higher, with a given PSA concentration, than in the case without interfering antibodies. However, unlike the standard measurement, in this case, the measurement of Dodiff31 or of ΔDO¹ allows detecting the presence of the interfering molecule. Hence, it is possible to apply an alarm to avoid reporting an erroneous result.

FIG. 14 shows the differences in optical density (mDO) of the Dodiff₃1 respectively with and without interfering antibodies.

The method of the invention may be carried out in a device of a known type, where necessary, adapted by means of knowledge of a person skilled in the art.

1. A method for assaying a target analyte in a biological sample in a liquid medium, comprising the following steps:
a. contacting the biological sample with first magnetic particles bearing a first receptor specific to a first site of attachment of the target analyte so as to form first complexes by bonding of the first magnetic particles with the target analyte this contacting being accompanied, when an interfering analyte is present in the sample, with the formation of interfering complexes by the non-specific bonding of said interfering analyte to the first magnetic particles;
b. applying a first magnetic field, and maintaining it, so as to locally combine all of the complexes formed in step a., and, if applicable, agglomerate interfering complexes with one another to form interfering aggregates;
c. negating the first magnetic field applied in step b. and adding in the liquid medium second magnetic particles bearing a second receptor specific to a second site of attachment of the analyte target;
d. measuring a first quantity representative of the amount of interfering aggregates in the liquid medium, to identify the presence or absence of said interfering aggregates;
e. applying a second magnetic field so as to form second complexes by bonding the first complexes with second magnetic particles; and
f. measuring a second quantity representative of the collective amount of interfering aggregates and of second complexes in the liquid medium so as to determine the amount of second complexes formed in step e. as a function of the first quantity for deducing therefrom the amount of target analyte present in the biological sample and, if applicable, the amount of interfering analyte.

2. The assay method according to claim 1, wherein step f. comprises calculating the difference between the second quantity measured at this step f. and the first quantity measured in step d.

3. The method according to claim 1, wherein steps c. and d. are performed substantially simultaneously.

4. The method according to claim 1, wherein step d. is carried out after negation of the first magnetic field, before adding second magnetic particles in the liquid medium.

5. The method according to claim 1, wherein holding the magnetic field applied in step b. lasts less than 5 minutes, preferably less than 3 minutes.

6. The method according to claim 1, wherein applying the second magnetic field includes a magnetisation at 8 mT for 1 second, followed by three successive sequences of magnetisations and cuts as follows: 15 mT for 60 seconds, 0 mT for 28 seconds, 8 mT for 1 second, 0 mT for 1 second.

7. The method according to claim 1, wherein measuring the first and/or second magnitude is selected from the group consisting of measurement by turbidimetry, measurement by nephelometry and measurement by counting.

8. The method according to claim 1, wherein step a. includes adding a diluent so as to dilute the biological sample, the ratio between the sample and the diluent being greater than or equal to 1:10 (volume).

9. The method according to claim 1, wherein step b. includes extracting the first complexes from the liquid medium.

10. The method according to claim 1, wherein step b. includes a sub-step b1. comprising removing the liquid medium followed by adding a reaction buffer, and preferably at least one washing between the removal of the liquid medium and the addition of the reaction buffer.

11. The method according to claim 10, wherein the reaction buffer comprises at least one anti-aggregation agent and/or a cell lysing agent.

12. The method according to claim 1, wherein the sample is a whole blood sample.

13. The method according to claim 1, wherein the interfering aggregates are identified when the first quantity exceeds a predefined threshold quantity.

14. A device arranged to carry out the method according to claim 1.
